(19) United States
(12) Patent Application Publication
(10) Pub. No.: US 2018/0030396 A1
(43) Pub. Date: Feb. 1, 2018

(54) SAMPLING DEVICE AND COMBINATION OF SAMPLING DEVICE AND SAMPLE VESSEL

(71) Applicant: THERMO FISHER SCIENTIFIC OY, Vantaa (FI)

(72) Inventor: Lauri PELTOSAARI, Espoo (FI)

(21) Appl. No.: 15/551,931

(22) PCT Filed: Feb. 19, 2016
(86) PCT No.: PCT/FI2016/050109

§ 371 Date: Aug. 18, 2017

(30) Foreign Application Priority Data
Feb. 19, 2015 (FI) 20155110

(51) Int. Cl. C12M 1/30; B01L 3/18; G01N 1/04

(52) U.S. Cl. CPC C12M 33/02; G01N 1/04; B01L 3/18; G01N 2001/028

(57) ABSTRACT

This invention relates to a sampling device comprising a shaft and a sample collection portion removably connected with the shaft, wherein the connection site between the shaft and the sample collection portion is weakened. The invention relates also to a combination of a sampling device comprising a shaft and a removable sample collection portion and a sample vessel configured to receive sample collection portion from the shaft.

### FIELD OF THE INVENTION

The present invention relates to a sampling device and a combination of a sampling device and a sample vessel.

### BACKGROUND

Disposable plastic loops and spreaders for inoculating microbial culture medium with a sample suspected of containing microbes and for selecting (“picking”) colonies of microbes from the surface of a culture plate are known in the art. Such tools (devices) are often formed of moldable synthetic resins, such as polystyrene or polypropylene and are generally shaped as a rod having a loop, T-shape, L-shape, needle, scoop, paddle of hook on one of the rod's ends. It is also known to use inoculation loops for transferring liquid microbial samples or cultures.

Prior manual methods of transferring microbial samples (such as cultivated bacteria, fungi, yeast) from an agar plate into a sample vessel require the user to smear the sample laden loop tip against the inside walls of the sample vessel and visually evaluate whether a sufficient amount of sample material has been transferred. This method is suboptimal because the shape of the sampling tip typically does not allow adequate contact of the sample material with the inner surfaces of the sample vessel. Further, the consistency of the sample materials, such as bacterial mass, is often slimy with a tendency to stick to the loop tip. This results in unpredictable sample volume and thus inaccurate result of the test.

To avoid contaminating the equipment, samples and personnel, aseptic measures must be taken. This means that sample transfer devices must be sterile and/or sterilized prior to and after use and between plates. One common means used for sterilization is flaming using a Bunsen flame. Alcohol may be used to facilitate the burning process. Metal or glass devices are compatible with flaming and can be used multiple times as they are heat tolerant, but there are some drawbacks. Sterilization is an additional time-requiring step and devices must be cooled before use so it is usual to use two devices so that the other may cool whilst the other is in use. The use of a burner and alcohol increases costs and presents a safety risk. Glass devices are also easily breakable.

Thus there is still a need of improving the efficiency and accuracy of the sampling.

### SUMMARY OF THE INVENTION

Both FDA (US Food and Drug Administration) and MDD/IVD standard ISO 13485 require that patient results generated by in vitro diagnostic (IVD) instruments must be traceable back to the original patient samples and to all analytically critical components of the analyzer systems including liquid consumables and disposables used in the analyzing processes.

In the context of the present invention a microbiology laboratory has several rooms or laboratories for different kinds of sample types. These rooms are also categorized based on biosafety levels (BSL) of the biological agents handled to BSL-1, BSL-2, BSL-3, and BSL-4. Many hazardous microbes, such as mycobacteria for example, are processed in BSL-3 level laboratories and non-hazardous microbes are processed in BSL-1 level laboratories. Samples from these laboratory rooms are transferred to a separate analyzing room for analysis, which analyzing room is equipped with suitable analyzer instruments and serve several different laboratory rooms.

Generally, the samples to be analyzed are prepared in a sample preparation station located at the BSL room or rooms. The sample preparation station comprises an instrument to facilitate movement of primary samples (e.g. bacterial colony) from original sample media (e.g. agar plate, blood culture bottle) to a sample vessel in an ergonomic manner which further reduces error.

As an example, a primary sample in an agar plate is taken to the sample preparation station and identification data of the primary sample which is stored in a barcode, radio frequency identification (RFID) tag or other indicia, is read to the sample preparation station with a suitable reader. Once confirmation of the data input is received, for example by a visual or audio signal, a specimen sample is taken from the primary sample with the sampling device of the invention, and the primary sample is returned to its storage. Next a sample vessel is taken, and its identification data is read to the sample preparation station with a suitable reader as was done with the primary sample. Then the sample in the sampling device (or sampling tool) is inserted in the sample vessel, and the end of the sampling device containing the sample is detached inside the sample vessel by twisting the sampling device, for example. The remaining part of the sampling device is discarded and the lid or cap of the sample vessel is closed. The sample vessel together with the sample is placed in an empty position of a sample vessel rack located in the sample preparation station in a proper orientation. The sample preparation station detects the location of the newly inserted sample vessel in the sample vessel rack and connects identification data obtained from the primary sample and the new sample vessel to the location of the sample vessel in the sample vessel rack.

When the sample vessel rack contains the required number of sample vessels, the sample vessel rack is removed from the sample vessel preparation station. In one example, the removal action also activates a locking means in the sample vessel rack. The locking means prevent the removal of any of the sample vessels located in the rack when locking is activated. The sample vessel rack is then inserted into an analyzing instrument, which insertion action optionally deactivates the locking means in the sample vessel rack, allowing removal of the sample vessels from the sample vessel rack for analysis operations.

The obtained analysis results are connected to the identification data of the sample from the sample preparation station, so that each analysis result can be tracked to a single sample.

The present invention provides a sampling device and a combination of a sampling device and sample vessel which can be used in the above described process.

First aspect of the invention is a sampling device. According to the present invention said device comprises

(a) a shaft or a handle and a shaft; and

(b) a sample collection portion removably connected with the shaft, wherein the site between the shaft and the sample collection portion is weakened in order to facilitate detachment of the sample collection portion upon twisting or bending or other excursion of force upon the shaft.

The second aspect of the invention is a combination comprising:

(a) a sampling device comprising a shaft and a removable sample collection portion; and

(b) a sample vessel.

According to the present invention the sample vessel is configured to receive and assist detaching the sample collection portion from the shaft.

### SHORT DESCRIPTION OF THE DRAWINGS

FIG. 1 shows three alternative embodiments of the sampling device (sampling tool) according to this invention. The sample collection portion comprises a loop (1A; front and side view), a hook (1B) or a scoop (1C) A “loop tool”, side & front views; B “hook tool”, C “scoop tool”.

FIG. 2 shows an exemplifying embodiment of a sampling device with a shaft that can be inserted to a reusable handle equipped with a plug-in system for fixing and releasing the shaft of the device.

FIGS. 3A-3D show one embodiment of a combination of a sampling device and a sample vessel (a kit).

FIG. 4 shows one embodiment of the sample vessel.

FIG. 5A-C shows three alternative embodiments of the sample collection portion with two level surfaces formed on both sides of the portion thereby forming an edge between the leveled surfaces and roundable outer surfaces of the portion.

FIG. 6 shows one embodiment of the sample vessel having protrusions in the inner side of the sample vessel.

### DETAILED DESCRIPTION

In one aspect, the invention provides a sampling device for the transfer of microbes comprising a shaft configured to allow removing of a sample collection portion by twisting or bending or other means; and a sample collection portion removably connected with the shaft. The device is suitable for both manual use and use as a part of an automated sampling system. After a sample has been picked up into the sample collection portion of the device, said portion is inserted into the sample vessel. The vessel and/or the shaft of the sampling device are then manipulated, for example by twisting or bending to break off the sample collection portion which is then left in the sample vessel with the collected sample material. Further processing, such as sample dilution and mixing, to separate the sample material from the collection portion is then performed. Essentially all the microbial material collected will be transferred to the vessel as the sample collection portion remains in the sample vessel.

The connection site between the shaft and the sample collection portion is configured to enhance breakage of the connection by twisting (rotation), bending or other means and thereby detach the sample collection portion and the shaft. In automated use it is also possible to break the connection by e.g. cutting or dragging. Removal by bending can be enhanced e.g. by modifying the shape of the sample collection portion and/or alignment of the sample collection portion from perpendicular to a slight angle in relation to the longitudal axis of the shaft. The shaft and the sample collection portion may be formed separately and fused or joined using known methods. Alternatively the device may be manufactured in one piece with a frangible connection between the shaft and the collection portion as is known in the art.

Breaking the connection between the portions can be effected, for example, by a rotational twist around the lengthwise axis of the shaft, by bending the shaft portion towards to the sample collecting portion or by other means of exerting force upon the frangible connection site. The forces needed for breaking the connection should allow manual use of the device. Suitable breaking force for rotational twist depends upon a number of factors including the shape and dimensions of the cross section of the shaft and the material(s) used to make the device. As a non-limiting example a shaft configured for gripping manually and having a diameter of about 4 mm should be breakable on applying a twisting force of 30 milli newton-meters (mN·m) or less in order to be easy to use. A shaft configured for gripping by hand or for automated use allows using twisting forces up to 6 N·m. Forces less than 4 N·m, preferably less than 2 N·m, are preferred. The weakened connection site should resist bending forces of approximately 21 mNm when the sample collection portion is bend around the center of the weakened connection site with a force acting on the sample collecting portion, the direction of which force is perpendicular in relation to the plane of the opening of the sample collection portion.

In manual use the rotational angle needed for breaking the connection (connection site) by rotational twist should not exceed 720 degrees (two rounds); preferably the angle should be less than 360 degrees, more preferably less than 180 degrees and most preferably less than 100 degrees.

The shaft is configured to allow collecting a sample from a liquid, semisolid or solid medium or surface. Optionally at least a portion of the shaft is flexible, and a portion of the shaft is rigid. Alternatively, the shaft is entirely flexible. Flexible shaft improves the user ergonomics. In addition, flexible shaft does not destroy the semisolid (e.g. agar medium or a tissue) surface of the sample growth medium by reducing the force directed on the surface when picking the sample.

Preferably the shaft portion of the device should allow the user to reach a bending angle (the angle between the ends of the longitudinal axis after bending) of at least 20 degrees, preferably at least 30 degrees and more preferably at least 40 degrees without breaking the shaft. Most preferably the shaft portion can be bent until about 80 degrees without breaking the shaft portion.

The elastic constant of the shaft portion can be 50 to 500 mm/N, preferably 100 to 300 mm/N and more preferably 120 to 200 mm/N. It is evident to a person skilled in the art that some portions, such as the gripping portion, of the shaft may be essentially rigid whereas the portion closer to the sample collecting end may be more flexible.

Optionally the shaft is grooved. Lengthwise grooves are preferred. They stiffen the shaft and improve the gripping when twisting the device. Shaft material savings are also obtained which reduces manufacturing costs.

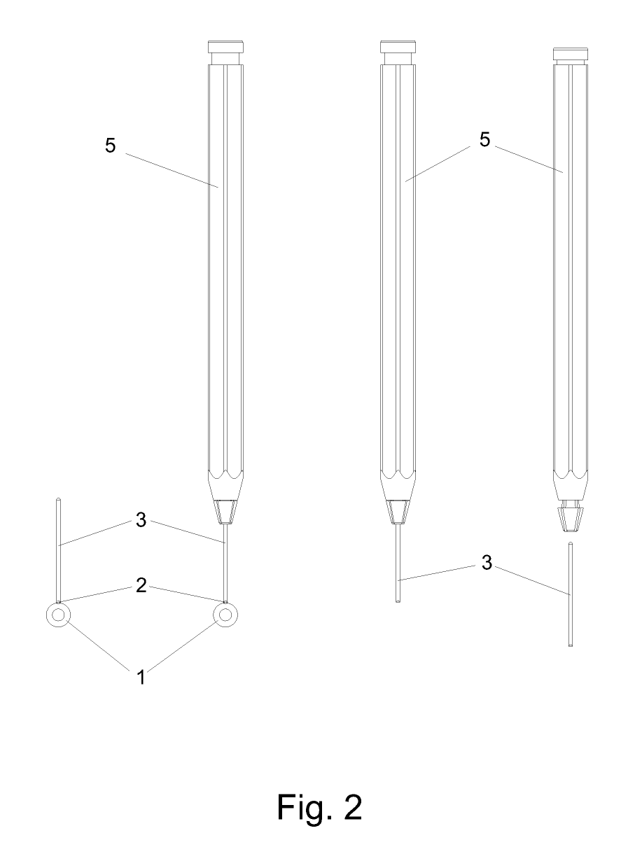

Alternatively the shaft is formed for insertion and fixing to a separate handle (5). In such an embodiment the shaft can be a simple stem extending from the connection site (2) in FIG. 2. This results in material savings and the sampling devices can be separately sterilized. The shaft part is still preferably long enough to avoid contamination of the sample, inoculation medium and the handle.

In one embodiment the shaft part may be connected to the reusable handle using the type of mechanisms utilized in pipettes to attach and remove pipette tips. Also other systems known from the art may be utilized. It is important that the shaft and handle parts are not able to rotate against each where twisting movement is used to detach the sample collection portion from the shaft, and that inserting and removal of the shaft remains aseptic to prevent any contaminations.

Between the shaft and sample collection portion there is a connection site (2) that is configured to yield and ultimately break as a response to twisting, bending or other manipulation of the shaft portion whereby the likewise rotation or bending of the sample collection portion has been prevented, thereby releasing the sample collection portion from the shaft. The connection site is able to withhold normal use of the device (streaking) but it is possible to remove the sample collection portion by a light twisting move or bending against a stopping surface.

The connection site can be weakened by a narrowing or perforating e.g. by applying plurality of small holes to the area to enhance breaking the connection (between the shaft and sample collection portion) by twisting, bending or other means. The narrowed or perforated portion reduces the resistance to rotational movement or bending when the collection portion is to be is detached. Alternatively, a material or a combination of materials amenable to breaking using manual force may be used to form at least the connection site of the sampling device.

In one embodiment a device with a weakened connection site is achieved by using 2-component molding of a one-piece sampling device.

The sample collection portion removably connected to the shaft may have any form suitable for collecting a sample containing microbes. It may comprise a loop, a needle, a hook or a scoop/paddle. Loops (or loop eyes) can be used to pick up solid bacterial mass or even liquid material if the surface tension of the material is strong enough to withhold the liquid inside the loop. It is possible to manufacture loops of different volumes, for example 1, 2 or 10 μl calibrated standard loops, which facilitates semi-quantitative or quantitative procedures such as making of serial dilutions. Scoop or spherical shaped sample collection portions (sample collectors), brushes or hooks are usable for collecting samples from solid or semisolid medium or surface. The size of the sample collection portion can be adapted to the needs (such as amount and state) of the sample.

The surface of the sample collection portion may be smooth, grooved or porous depending on the type of the sample. For collecting solid or semisolid microbial, such as bacterial, samples the material of the sample collection portion should be rigid enough to allow collecting the sample. In one embodiment the sample collection is not porous and thus does not adsorb the sample. In one embodiment of the invention one, two or more level surfaces are formed on side(s) of the sample collection portion. Together with the other surfaces (which have any form, including leveled and roundable surface) an edge between two parallel surfaces is formed. The edge can be used to collect samples with are attached to a surface, such as a surface of semisolid culturing media. The edge allows collecting a sample by scratching. In an alternative embodiment an edge suitable for collecting a sample by scratching is introduced to the sample collection portion in form of a protrusion or a bracket.

This type of edged sample collection portion is especially usable in collecting samples from semisolid or solid surfaces.

The sample collection portion can be configured and sized to be suitable for a sample to be collected.

The present invention is also directed to a combination (a sampling kit) comprising a sampling device, preferably the sampling device as described above and a sample vessel. In one embodiment the sample vessel is configured and sized to be suitable for receiving a sample collection portion of the sampling device and to enhance breaking the connection between the shaft and the sample collection portion.

In one embodiment the vessel is configured to limit the movement (e.g. the twisting, bending, etc.) of the sample collection portion thereby enhancing breakage between the shaft and the sample collection portion. This can be achieved by e.g. inner surface providing friction to the movement of the sample collection portion, using asymmetric inner surface or by a shape configured to resist free twisting of the sample collection portion. Alternatively the vessel may have a form comprising a slot enhancing the breaking of the connection by bending. In one embodiment the vessel, especially the vessel body, is essentially rigid and does not allow e.g. squeeze using fingers in such degree that the inner walls of the vessel body deform inwards and aid in the detachment of the sample collection portion. Preferably the vessel body does not deform using a force of 60 N or less.

An exemplary sample vessel of the invention comprises an elongated vessel body for containing the sample wherein the cross-section of the vessel body on a transversal plane in relation to the lengthwise center axis of the vessel body the inner surface of the vessel body is rotationally asymmetrical in relation to the lengthwise center axis. The asymmetrical shape of the inner surface of the vessel body allows for easy and secure insertion of a sample in the sample vessel with a sampling device here described by wedging the sample collection portion of said device against the inner surface(s) of the vessel body and by twisting (rotating) or bending the shaft of the said device and thus breaking the sample collection portion (end portion, lower portion, next to the connection site) of the shaft together with the sample inside the vessel body.

In one embodiment of the sample vessel the cross-section of the inner surface of the vessel body is substantially oval-shaped. The oval shape of the cross-section extended to the outer surface of the vessel body enhances the orientation of the sample vessel when placed in a sample vessel rack for the automatic handling.

In an embodiment the inner surface of the sample vessel comprises one or more protrusions, preferably at the bottom area of the sample vessel, which protrusion(s) form a narrow slot inside the sample vessel. The formed slot can be used as an additional support surface(s) for detaching a part of a suitable sampling device, for example detaching the shaft and the sample collection portion.

In one embodiment the sample collection portion and protrusion(s) in the inner side of the sample vessel are be configured and sized to be suitable for a detachment of the sample collection portion by rotational twisting or bending. In one embodiment the sample collection portion is set in the area of the slot so that it can be detached from the shaft by twisting. Protrusions are especially beneficial when the sample collection portion is small of size and thereby would without protrusions easily rotate thereby making the detachment difficult. Small size of the sample collection portion is usually beneficial for further processing of the sample and for example does not prevent mixing and dissolution of the sample. Example of this type of vessel and vessel rack are disclosed in a patent application filed at the same date and by the same applicant as the present application.

The shaft (3), the sample collection portion (1), the sampling device (5) and the sample vessel (6) jointly or independently can be formed of a polymer, including, for example, plastic polymers such as polypropylene (PP), polyethylene, polyester ketone (PEEK), poly(methyl methacrylate (PMMA), polystyrene (PS), polycarbonate (PC) or a mixture thereof. Also various composite materials are usable, e.g. composite of glass and plastic polymer. The polymeric portions can be made by injection molding, extrusion or casting. Preferred material, especially at the connection site (2) between the shaft and the sample collection portion, should be fragile enough in order to allow separating the portions by an axial twisting movement but still at the same time be flexible enough in axial direction to allow picking the sample. Forces needed to separate the portions must be minimized in order to make the device as ergonomic as possible but the connection site must be strong enough to withhold axial bending of the device during sample pick up and to prevent accidental breaking of the portions.

Pre-sterilized plastic single-use disposable sampling devices have several advantages. The inoculation process is faster as some of the sterilization steps may be omitted. Plastic inoculation devices can also be made flexible to facilitate streaking without damaging the gel-like target medium surface. Typically plastic devices are either heat sterilized and, consequently, destroyed and disposed, or collected aside for a later sterilization in an autoclave and disposal. Alternatively the polymeric material is biodegradable. The shaft and/or the sample collection portion may also be formed completely or partially of metal or glass. The shaft and the sample collection portion may also be formed of different materials, i.e. the other may be formed of a composite material of two or more different polymers or polymer and one or more other materials, for example metal, glass or magnetic material. In one embodiment shown in FIG. 2 a reusable handle (5) is made of glass and a disposable sampling device is made of polymeric material.

Preferably the shaft is formed as one piece and the sample collection portion is formed of one piece. Optionally the sampling device as a whole is formed of one piece. One piece formation reduces manufacturing and assembly costs.

The finished sampling device is preferably a one piece instrument but it may be manufactured in more than one step, for example using multi-component injection molding using two or more different materials. When the shaft and the sample collection portion are molded in separate phases the interface between the portions, the connection site, will intrinsically form weaker than compared to one-component molding. This weaker connection site allows breaking off the sample collection portion by light twisting, bending or other movement. A multi-component injection molded sampling device may also have a sample collection portion formed of material that is chemically inert to the sample or any subsequent chemical used during processing of the sample and a shaft formed out of material that is chemically less inert, but has other desirable properties such as easier breakability or lower price.

The sample collection portion is preferably chemically inert to the sample or any subsequent liquid in contact with it. Alternatively the collection portion can be formed of material soluble to a liquid added after the picking step but it is also preferable that the sample collection portion material does not interfere with any subsequent analyses performed to the sample. Optionally the sampling device, the shaft and/or the sample collection portion are/is coated in order to improve surface properties of the device, shaft or portion. The sample collection portion material or its coating may act with the sample chemically or mechanically, for example by virtue of filtering materials. A person skilled in the art understands that the needs for the surface and material of the device and especially the sample collection portion are dependent on the type of the samples, culture media used and subsequent analysis steps.

### DESCRIPTION OF AN EMBODIMENT WITH REFERENCES TO DRAWINGS

Reference is now made to Figures which show a one-piece embodiment of the sampling device (5). In the figures is shown a shaft (3) and a sample collection portion (1). A connection site (2) between said shaft and sample collection portion is breakable using manual force. The exemplifying embodiment disclosed of the figures is configured for manual use and so called pencil gripping. The gripping portion (upper end, gripping end) (4) of the shaft has a diameter of about 4 mm and length of 115 mm. The shaft comprises lengthwise grooves in order to improve gripping and/or increase stiffness. Lengthwise center axis is shown in FIG. 2C. Also various cross or star like cross sections known in the art are useful embodiments.

In alternative embodiments the shaft may be longer or shorter; cross section and material of the shaft may vary within desired use (such as finger gripping, hand gripping or automated use).

In an embodiment the cross section of the portion end of the shaft (3) gradually narrows towards to the connection site (2) with the sample collection portion (1). Optionally said lower portion of the shaft can be uniform and have a cross section similar to or different from the gripping portion. In one embodiment the lower portion of the shaft is flexible.

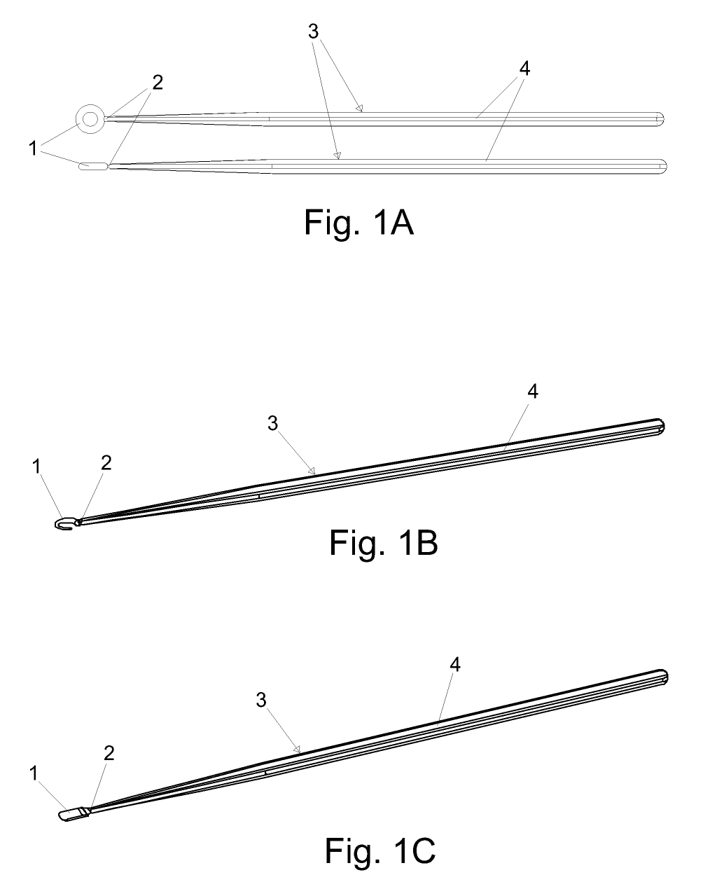

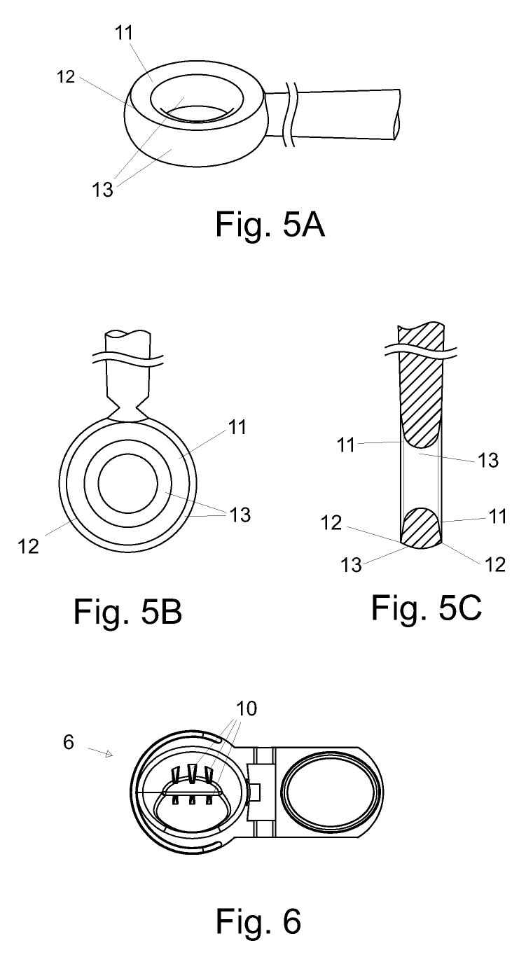

A loop shown in FIG. 1A; a paddle of hook shown in FIG. 1B and a scoop shown in FIG. 2C are examples of alternative embodiments of the sample collection portion (1). Essentially flat side section and narrowed connection site (2) (jointly and independently) enhances the detachment of the collection portion by rotational twisting. Respectively FIGS. 5A, B, C show embodiments of the sample collection portions with level surfaces (11) forming an edge (12) with other surfaces (13) of the portion.

FIG. 2 illustrates an embodiment where the shaft (3) is formed to be releasably fitted and fixed to a separate handle. The fixing should allow detaching the sample collection portion and the shaft by twisting or bending the handle. Then, according to one embodiment, the shaft portion can be released from the handle

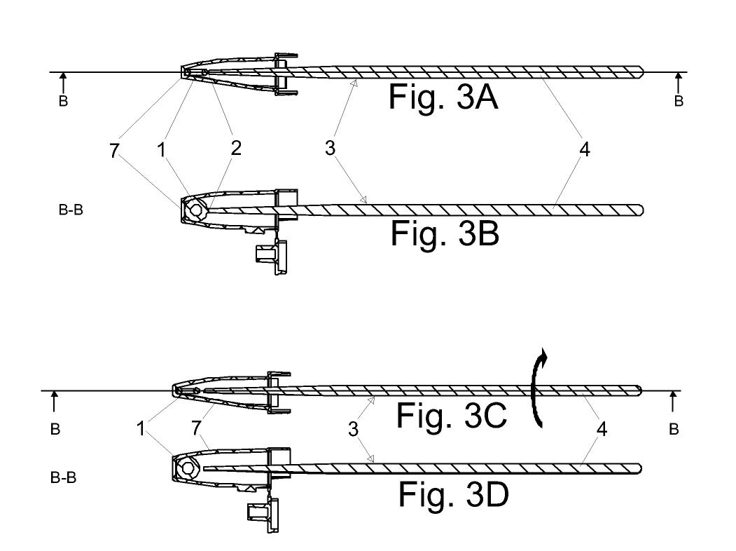

FIGS. 3A, B, C, D show one embodiment of combination of a sampling device (5) and a sample vessel (6). Said combination can also be called a sampling kit. Rotational twist for breaking the connection between the sample collection portion and the shaft is shown in FIG. 3C.

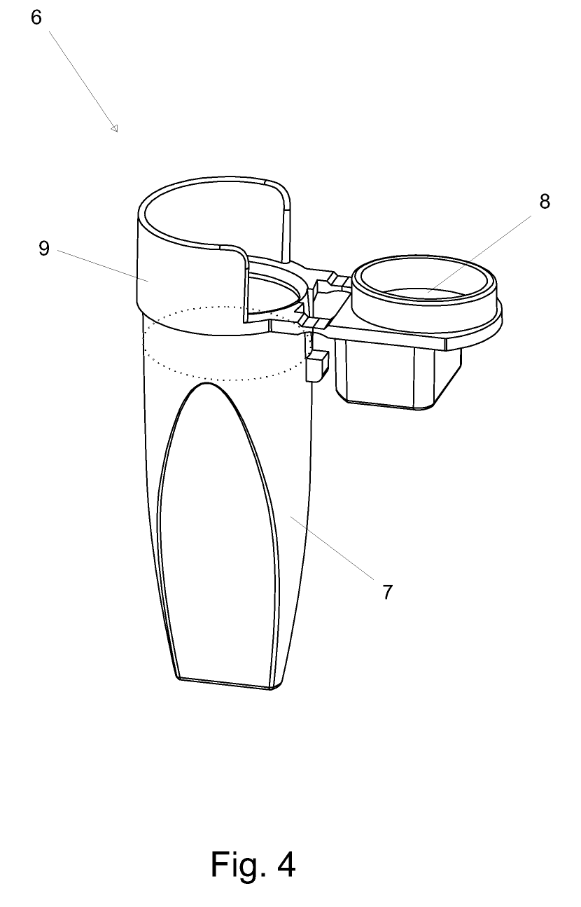

In FIG. 4 is shown one embodiment of a sample vessel (6) of the combination or kit here described. The vessel comprises an elongated vessel body (7) and as an optional feature a cap (8) attached to the upper end of the vessel body with integral hinges. The outer edge of the cap (8) is partially surrounded with a splash guard (9) connected to the upper end of the vessel body. FIG. 6 shows an alternative embodiment of the vessel having protrusions (10) in the inner side of the vessel. The number of the protrusions is not limited but typically is two, three or four protrusions.

The elongated vessel body in this embodiment has an oval cross-section, both for the outer and inner surface of the vessel body. The oval cross-section of the inner surface of the vessel body allows the sample collection portion of the sampling device (end portion of the device/tool) to be twisted or bend between inner surfaces of the vessel body, so that the end of the sample collecting device can be broken off by rotationally twisting the sample collecting device, and the sample collection portion of the sampling device is left together with the sample inside the vessel body. A rotational twisting also reduces the sticking of the microbial sample to the inner walls of the vessel and thus enhances the dissolution of the sample. The oval cross-section of the outer surface of the vessel body enhances the usability of the sample vessel in automated and robotic handling of the sample vessel. Also essentially rigid shape of the vessel is beneficial for automated processes.

It should also be understood that the device and the combination (kit) of the present invention can be used with various types of microbial samples. Wherever the culturing of cells or microorganisms is required, such as with food stuffs, water supply systems, clinical or microbiological samples, the device of the present invention may be used.

It is to be understood that the terminology employed herein is for the purpose of description and should not be regarded as limiting.

The features of the invention described here as separate embodiments may also be provided in combination in a single embodiment. Also various features of the invention described here in the context of a single embodiment, may also be provided separately or in any suitable sub-combination. It should be understood, that the embodiments given in the description above are for illustrative purposes only, and that various changes and modifications are possible within the scope of the disclosure.

1. A sampling device comprising:
(a) a shaft; and
(b) a sample collection portion removably connected with the shaft, wherein the connection site between the shaft and the sample collection portion is configured to enhance breakage of the connection by twisting (rotation) or bending.

2. The device according to claim 1 wherein the connection site is weakened by narrowing.

3. The device according to claim 1, wherein the sample collection portion comprises a loop, a needle, a hook, a scoop or a paddle.

4. The device according to claim 1, wherein the sample collection portion comprises a loop.

5. The device according to claims 1 made of two or more materials and/or two or more portions.

6. The device according to claims 1 made of polymeric material.

7. The device according to claim 5, wherein the polymeric material is polypropylene (PP), polyethylene, polyester ketone (PEEK) or a mixture thereof.

8. The device according to claim 5, wherein the shaft is made of metal, glass or plastic-polymer composite material.

9. The device according to claim 1 wherein at least the sample collection portion is disposable.

10. The device according to claim 1, wherein the shaft is inserted to a resuable handle.

11. The device according to claim 1, wherein the device is made of one piece.

12. The device according to claim 11 which is formed by two-component molding.

13. A combination comprising:
(a) a sampling device comprising a shaft and a removable sample collection portion; and
(b) a sample vessel configured to receive and assist detaching the sample collection portion from the shaft,
wherein, upon placement of the sample collection portion into said sample vessel, the sample collection portion is detached from the shaft by rotating or bending the shaft such that the sample collection portion remains within the vessel.

14. A combination according to claim 13, wherein the sample vessel is essentially rigid.

15. The combination according to claim 13 wherein the device is a device according to claim 1 and the sample vessel is configured to limit the movement of the sample collection portion.
